(19) United States
(12) Patent Application Publication
(10) Pub. No.: US 2013/0072451 A1
(43) Pub. Date: Mar. 21, 2013

(54) Low Viscosity Personal Care Composition

(71) Applicants: Qiaolu LU, Beijing (CN); Paul (Robert) Tanner, Lebanon, OH (US); Robert (Bao Kim) Ha, Hamilton, OH (US)

(72) Inventors: Qiaolu LU, Beijing (CN); Paul (Robert) Tanner, Lebanon, OH (US); Robert (Bao Kim) Ha, Hamilton, OH (US)

(21) Appl. No.: 13/616,589

(22) Filed: Sep. 14, 2012

(30) Foreign Application Priority Data
Sep. 16, 2011 (CN) AA-825F

(51) Int. Cl. A61K 8/81; A61Q 19/00; A61K 8/891

(52) U.S. Cl. 514/26; 514/772.6; 514/563; 514/99; 514/636

(57) ABSTRACT

A low viscosity personal care composition comprising from about 10 % to about 99% of water, from about 0.01% to about 20% of a suspended particulate material, from about 0.01% to about 5% of an acrylic polymer, wherein the composition has a viscosity of from about 80 to about 1000 mPa·s, and a pH of from about 4 to about 9, wherein the acrylic polymer provide a selection yield value of at least 0.11 Pa. An array of two or more low viscosity personal care compositions, wherein one of the personal care compositions comprises said particulate material at a level different from those in the other personal care compositions.

### CROSS REFERENCE TO RELATED APPLICATIONS

This application claims the benefit of PCT China Application No. PCT/CN2011/0797 filed Sep. 16, 2011.

### FIELD OF THE INVENTION

The present invention relates to a low viscosity personal care composition comprising a suspended particulate material. The present invention also relates to an array of two or more of the low viscosity personal care compositions.

### BACKGROUND OF THE INVENTION

Toner, also known as “clear lotion”, is a low viscosity personal care composition comprising essentially no emulsifiers, Toner is typically applied to skin after a facial cleanser is used and before a skin moisturizer is used. The use of toner has long been deemed as a supplementary step in the skin care regimen to neutralize the elevated pH of the skin surface which is caused by the facial cleanser, and thereby prepare the skin for better absorption of the moisturizer.

In recent years, the use of a toner has been designed as an independent step in the regimen, and various benefits that a toner can provide are being explored.

It is desirable that a toner has a translucent appearance. Some consumers tend to believe a translucent toner is “richer” than a transparent toner. In order to achieve a translucent to opaque appearance in a personal care product, emulsification techniques have been traditionally used. An emulsified system comprises a water phase and an oil phase. The mechanism behind achieving a translucent appearance in an emulsion is that the well-dispersed small oil droplets have a refractive index that differ from that of water, which in turn causes the light to reflect at the interface of the oil and aqueous phases. This light reflection leads to translucency of the emulsion.

However, toner has a simpler structure than that of an emulsion. Since the toner remains on the skin and is not rinsed off, it is desirable that the emulsifier level in the toner is kept as low as possible, Besides, the toner should preferably have a low level of oil to prevent an oily skin feel.

In addition to translucent appearance, it is also desirable for the toner to provide a fresh and smooth skin feel. It is known that certain particulate materials, for example silicone resins, are used for this purpose. For instance, WO 00/56272 A1, which is assigned to Color Access, Inc, relates to gel-like liquid powder compositions comprising silicone resins. However, the creation of a translucent appearance and a fresh silky skin feel should not compromise the low viscosity requirement of the toner, which is less than 1000 mPa·s. Higher viscosities, e.g. greater than 1000 mPa·s are characteristic of gel-like composition, which are distinct from toners.

Hence, it is desirable to provide a mild, low viscosity personal care composition, which has a translucent appearance, provides a fresh skin feel, has essentially no emulsifier, and is stable. None of the prior published references discloses the present invention and meets the benefits of the present invention.

### SUMMARY OF THE INVENTION

In a first aspect, the present invention relates to a low viscosity personal care composition comprising:

a) from about 10% about 99% of water,

b) from about 0.01% to about 20% of a suspended particulate material,

c) from about 0.01% to about 5% of an acrylic polymer, wherein the composition has a viscosity of from about 80 to about 1000 mPa·s, and a pH of from about 4 to about 9, wherein the acrylic polymer provide a selection yield value of at least 0.11 Pa.

In a second aspect, the present invention relates to a low viscosity personal care composition comprising:

a) from about 50 % to about 99 of water,

b) from about 0.01% to about 20% of polymethylsilsesquioxane,

c) from about 0.01% to about 5% of an acrylates/vinyl isodecanoate cross polymer,

d) from about 0.01% to about 10% of a viscosity reducing agent, which is a dissociable active agent, wherein the composition has a viscosity of from about 200 to about 600 mPa·s, and a pH of from about 5 to about 7.

In a third aspect, the present invention relates to an array of two or more low viscosity personal care compositions, wherein one of said personal care compositions comprises a suspended particulate material at a level different from those in the other personal care compositions.

### DETAILED DESCRIPTION OF THE INVENTION

It has been unexpectedly found that a refreshing, translucent. mild, low viscosity personal care composition (e.g. a toner) can he provided through stable suspension of particulate materials using select acrylic polymers as appropriate suspension aids, and through minimized usage of emulsifiers. The select acrylic polymers of the present invention provide a selection yield value of at least 0.11 Pa. The present invention is not limited to any particular mechanism of action, but is believed to operate by a crosslinked internal structure in the composition built by the suspension aid which supports the particulate materials in the composition and diminish the particulate material separation out of the composition. The present invention also provides an array of products with varied translucency levels, which meet different consumers' needs through varying the suspended particulate material level in the present composition.

While the specification concludes with claims particularly pointing out and distinctly claiming the invention, it is believed that the present invention will he better understood from the following description.

All percentages, parts and ratios are based upon the total weight of the compositions of the present invention, unless otherwise specified.

All the tests are conducted at 25° C. unless otherwise specified in the present specification.

As used herein, “comprising” means that other steps and other ingredients which do not affect the end result can be added. This term encompasses the terms “consisting of” and “consisting essentially of”.

As used herein, the term “suspended particulate materials” means the plurality of particles incorporated in the present aqueous composition do not separate in solid form from the composition. In other words, the particulate materials do not visibly settle to the bottom or visibly float to the top of the composition. The suspension stability can he tested through an accelerated stability test conducted at an elevated temperature, usually 40° C.

As used herein, the term “specific density” means the ratio of the density of a substance versus the density of water.

As used herein, the term “selection yield value” provided by the acrylic polymer means the yield value measured in a screen solution. The screen solution and the yield value measurement method are described in detail below.

As used herein, the term “dissociable active agent” means the active agents which carry positive and/or negative charges on its structure.

As used herein, the term “personal care composition” refers to a consumer product that is suitable for topical application on mammalian keratinous tissue.

As used herein, the term “array” means two or more products displayed on a shelf together, and/or sold together.

The elements of the present low viscosity aqueous composition are described in more detail below.

Suspended Particulate Materials

The present aqueous composition comprises from about 0.01%, about 0.1%, and about 1% to about 5%, about 10%, and about 20% of a suspended particulate material, or a mixture of various suspended particulate materials.

In the present invention, useful suspended particulate material comprises particles which are measured in microns. The particulate materials useful in the present invention have a median particle size and particle size distribution such that they are below the tactile perception threshold of most consumers, and yet are not so small as to he ineffective for a silky and smooth sensory. The median particle size refers to the particulate materials in a neat form, i.e., in an essentially pure, powder form. Median particle size can be determined by any suitable method known in the art, such as the ASTM Designation E20-85 “Standard Practice for Particle Size Analysis of Particulate Substances in the Range of 0.2 to 75 Micrometers by Optical Microscopy”, ASTM Volume 14.02, 1993. The particulate materials useful in the present invention can comprise particles having a median particle size of from about 1 μm, about 2 μm, and about 5 μm to about 10 μm, about 15 μm, about 20 μm, and about 40 μm. It is preferred that more than 50%, more than 60%, more than 70% of the particles of the particulate materials fall within the size ranges prescribed for the respective median values.

Useful particulate materials of the present invention can comprise particles having a specific density (at 25° C.) of from about 0.5, about 0.6, about 0.8, and about 0.9 to about 1.1, about 1.3, about 2.0, and about 3.0.

The particles of the particulate materials can be essentially spherical. Spherical particles comprise those particles having a smooth outer surface, and those particles which have small spikes protruding from their outer surfaces.

The particles of the particulate materials are essentially insoluable. In particular, the particulate materials have a solubility of no more than about 1 gram per 100 grams of water at 25° C., preferably no more than about 0.5 grams, more preferably less than about 0.1 grams,

Suitable particulate materials can he of a variety of chemical compositions, for example, nylon, silica, organosilicone polymers (e.g., polymethylsilsesquioxane), polyethylene, polyurethane, acrylate or methacrylate polymers or copolymers, polybutylene, polyethylene, polyisobutylene, polymethylstyrene, polypropylene, polystyrene, polyurethane, polyacrylonitrile, polyacrylic acid or derivatives thereof, polymethacrylic acid, polystyrene, polytetrafluoroethylene (PTFE), poly(vinylidene chloride), polyamide, and combinations thereof. Also useful are particulate materials made from mixed polymers (e.g., copolymers, terpolymers, etc.), such as polyethlene/polypropylene copolymer, polyethylene/propylene/isobutylene copolymer, polyethlene/styrene copolymer. and the like.

In one embodiment, the particles of the particulate materials are selected from a group consisting of polymethylsilsesquioxane, nylon, polyethylene and combinations thereof. The polymethylsilsesquioxane particulate materials are also commonly known as silicone resins.

Suitable commercially available examples of the particulate materials in the present compositions are silicone resins sold as Tospearl® series from Momentive Performance Materials Inc, including Tospearl® 2000, Tospearl® 145. Tospearl® 150, Tospearl® 1320 and the like.

Other commercially available examples are polyethylene sold as Microthene® FN 510-00 from Equistar, nylon sold as Nylon 12 from IMCD UK Ltd, polytetrafluoroethylene organic particulate material sold as FluoroPURE™ 100C from Shamrock Inc, ethylene/acrylic acid copolymer sold as EA-209 from Kobo and polyurethane (HDI/trimethylyol hexyl lactone) microsphere sold as BPD-500 from Kobo Products, Inc.

Acrylic Polymer and Selection Yield Value

The present composition comprises from about 0.01%, about 0.05%, and about 0.1% to about 1%, about 2%, and about 5% of an acrylic polymer.

An acrylic polymer is a polymer that has 50% or more residues of (meth)acrylic monomers, based on the weight of the polymer. As used herein and as defined by F. W. Billmeyer, in the Textbook of Polymer Science, second edition, published by Wiley-Interscience, 1971, the term “polymer” is a large molecule comprising a repetition of smaller, simpler chemical units, “Monomers” are chemicals that react with each other to become the repeated units of a polymer, Polymers may be linear, branched, crosslinked, star shaped, or any combinations thereof. Polymers may be homopolymers (i.e., built up from one type of monomer) or copolymers (built up from two or more types of monomer). Copolymers may have the monomer units arranged randomly, in blocks, in sequence, or combinations thereof. Polymers have a weight-average molecular weight of greater than or equal to 1,000, or even greater than or equal to 10000, Polymers may have any of a wide variety of physical forms, and some polymers may be converted from one physical form to another. Some polymers are solid, liquid, or a combination thereof. Some polymers are dissolved in a solvent to form a solution. Some polymers are dispersed: that is, they exist as polymeric particulate, materials distributed in a liquid medium.

A (meth)acrylic monomer is any monomer that is (meth)acrylic acid, any ester thereof, or an amide thereof. Esters or amides of (meth)acrylic acid can have alkyl groups, aromatic groups, any of a wide variety of substituent groups such as, hydroxyl, epoxide, amine, thiol, urethane, carboxyl, or other substituent groups, or any combination thereof. Some (meth)acrylic monomers have exactly one carbon-carbon double bond, while other (meth)acrylic monomers have two or more carbon-carbon double bonds. Some monomers are described herein as esters of (meth)acrylic acid with certain alcohols. It is to be understood that such a description refers to the structure of the monomer so described, which may he made by any method, including by reacting (meth)acrylic acid with that certain alcohol.

Acrylic polymers have long been used in various industries to provide rheology modifying benefits including but not limited to thickening capability as well as suspension capability for a composition in which it is incorporated. Some polymers are commercially advertised as focusing on providing suspension capability, while the other polymers are advertised as more focusing on building up viscosity. It is important for the present invention to select suitable acrylic polymers for the present low viscosity personal care composition, which can provide a high enough yield value to suspend particulate materials, while at the same time not increasing the viscosity too much.

It is surprisingly found by the present applicant that an acrylic polymer suitable for use as a suspension aid for suspending particulate materials in a low viscosity personal care composition can he selected through a screening process in a screen solution. The selection yield value provided by the acrylic polymer means the yield value for the screen solution incorporating a acrylic polymer to he screened. The screen solution can he prepared according to the following steps.

a) prepare an acrylic polymer solution by dispersing a predetermined amount of an acrylic polymer in water and adjusting the pH to about 6,

b) optionally, add a viscosity reducing agent to the acrylic polymer solution to reach a low viscosity aqueous composition, wherein the predetermined amount of acrylic polymer together with or without the viscosity reducing agent make the viscosity of the low viscosity Ligneous composition to he about 500±60 mPa·s.

The selection yield value is measured for each screen solution comprising a acrylic polymer to he screened. If an acrylic polymer renders a selection yield value of at least 0.11 Pa, preferably at least 0.12 Pa, it is identified as a suitable acrylic polymer for use in the present personal care composition to stably suspend particulate materials. Without being bound by theory, it is believed that a favorable intersection point is found between the thickening capability and the suspension capability for an acrylic polymer as a suitable suspension aid for the present personal care composition.

Suitable commercially available acrylic polymers for the present invention include acrylates/vinyl isodecanoate crosspolymer sold as Stabylen 30 from 3V Inc, acrylates/C₁₀₋₃₀ alkyl acrylate crosspolymers sold as Carbopol® 1382, Pemulen™ TR-2 and Pemulen™ TR-1 from Lubrizol, and carbomer sold as Carbopol® 981 and Carbopol® 980 and Carbopol® from Lubrizol.

Viscosity Reducing Agent

In order to obtain a viscosity of the present composition within the range of from about 80 mPa·s, or about 200 mPa·s to about 600 mPa·s, or about 1000 mPa·s, the present composition can include from about 0.01%, about 0.5%, and about 1% to about 2%, about 5%, and about 10% of a viscosity reducing agent.

A suitable viscosity reducing agent can he an electrolyte, preferably a monovalent salt, including but not limited to sodium chloride, potassium chloride, and combinations thereof. The monovalent salt can be intentionally incorporated into the composition, or it can also be present as an impurity from any other ingredient in the present composition. The viscosity reducing agent can also be a skin active agent dissociable in a personal care composition having a pH of from about 4 or 5 to about 7 or 9. Such a skin active agent dissociable in an aqueous composition includes dipotassium glycyrrhizinate sold as NET-DG from Barnet Products Corporation, undecylenoyl phenylalanine sold as Sepiwhite® MSH from Seppic S.A, sodium ascorbyl phosphate sold as Stay-C® 50 from DSM Nutritional Products, Inc, and hexamidine diisethionate sold as Elestab® HP 100 from Cognis.

According to the level of particulate materials to be suspended, the level of polymers, the specific viscosity and the particular skin feel requirements, the amounts and species of viscosity reducing agents can he determined accordingly. In one embodiment, where the product requires a particular translucency level or skin feel, the amount of required particulate materials can he thereby determined. As a next step, the viscosity reducing agent level together with the acrylic polymer level can be determined without undue effort, depending on a specific viscosity limitation of what is desired within the range of from about 80 mPa·s to about 1000 mPa·s.

Humectants

The present composition can comprise one or more humectants. A variety of humectants can be employed and each can be present at a level of from about 0.1%, about 1%, and about 2% to about 5, about 10%, and about 30%.

Nonlimiting examples of humectants include materials selected from a group consisting of polyhydroxy alcohols such as sorbitol, glycerol, hexanetriol, propylene glycol, butylene glycol, pentylene hexylene glycol, propoxylated glycerols and the like; polyethylene glycols; guanidine; glycolic acid and glycolate salts (e.g., ammonium and quaternary alkyl ammonium); lactic acid and lactate salts; aloe vera in any of its forms e.g., aloe vera gel); sugars and starches; sugar and starch derivatives (e.g., alkoxylated glucose); hyaluronic acid; lactamide monoethanolamine; acetamide monoethanolamine; and combinations thereof. In one embodiment, the humectant is a polyhydroxy alcohol.

Emulsifiers

The present composition can comprise an emulsifier useful for dispersing or suspending an oil phase, or oily compounds such as perfume within the aqueous phase. In one embodiment, however, the present composition comprises no more than about 10%, no more than 5%, no more than 1%, or no more than 0.5% of an emulsifier. In another embodiment, the composition of the present invention comprises no emulsifiers.

A wide variety of emulsifiers such as nonionic emulsifiers, anionic emulsifiers, cationic emulsifiers and amphoteric emulsifiers can be employed herein. Nonionic emulsifiers are preferred.

Non-limning examples of nonionic emulsifiers for use herein include: sugar esters and polyesters, alkoxylated sugar esters and polyesters, C₁-C₃₀ fatty acid esters of C₁-C₃₀ fatty alcohols, alkoxylated derivatives of C₁-C₃₀ fatty acid esters of C₁-C₃₀ fatty alcohols, alkoxylated ethers of C₁-C₃₀ fatty alcohols, polyglyceryl esters of C₁-C₃₀ fatty acids, C₁-C₃₀ esters of polyols, C_{1-C}₃₀ ethers of polyols, alkyl phosphates, polyoxyalkylene fatty ether phosphates, fatty acid amides, acyl lactylates, soaps, and combinations thereof. Non-limiting examples of other emulsifiers for use herein include polyethylene glycol 20 sorbitan monolaurate (polysorbate 20), steareth-20. ceteareth-20, PPG-2 methyl glucose ether distearate, ceteth-10, polysorbate 80, cetyl phosphate, potassium cetyl phosphate, diethanolamine cetyl phosphate, polysorbate 60. glyceryl stearate. PEG-100 stearate, polvoxyethylene 20 sorbitan trioleate (polysorbate 85), sorbitan monolaurate, polyoxyethylene 4 lauryl ether sodium stearate, polyglyceryl-4 isostearate, hexyl laurate. PPG-2 methyl glucose ether distearate. ceteth-10, diethanolamine cetyl phosphate, glyceryl stearate. PEG 40 hydrogenated castor oil, PEG-60 hydrogenated castor oil, glycereth-25 PCA isostearate, and combinations thereof.

Other Ingredients

The present composition can comprise a variety of other ingredients which are conventionally used in a given product type provided that they do not unacceptably alter the benefits of the invention.

In any embodiment of the present invention, the actives useful herein can he categorized by the benefit they provide or by their postulated modes of action. However, it is to he understood that the actives useful herein can in some instances provide more than one benefit or operate via more than one mode of action. Therefore, classifications herein are made for the sake of convenience and are not intended to limit the active to that particular application or applications listed.

Examples of these ingredient classes include: abrasives, absorbents, aesthetic components such as fragrances, pigments, colorings/colorants, essential oils, skin sensates, astringents, etc. (e.g., clove oil, menthol, camphor. eucalyptus oil, eugenol, menthyl lactate, witch hazel distillate), anti-acne agents (resorcinol, sulfur, salicylic acid, erythromycin, zinc, sebum control agents, e.g. Phlorogine® from Presperse Incorporated etc.), anti-aging agents including N-acetyl derivatives, e.g. N-acetyl-L-cysteine, thiols, e.g. ethane thiol; hydroxy acids (e.g., glycolic acid, lactic acid, and the like), phytic acid, lipoic acid; lysophosphatidic acid, skin peel agents (e.g., phenol and the like), vitamin B3 compounds (e.g. niacinamide, tocopherol nicotinate, and the like) and retinoids, antifoaming agents, antimicrobial agents (e.g., iodopropyl hutylcarbamate), antioxidants, biological additives, buffering agents, chelating agents, colorants, cosmetic biocides, denaturants, film formers or materials. e.g., polymers, for aiding the film-forming properties and suhstantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin soothing and/or healing agents (e.g., panthenol and derivatives (e.g., ethyl panthenol), aloe vera, pantothenic acid and its derivatives, allantoin, hisabolol, and dipotassium glycyrrhizinate), thickeners, and vitamins and derivatives thereof.

Method of Preparation

The present compositions can he prepared through conventional methods which are known in the art of making topical compositions. Such steps typically involve mixing of the ingredients in one or more steps to a relatively uniform state, with or without heating, cooling, application of vacuum, and the like.

For example. the present composition can be prepared according to the following steps: 1) prepare a first premix of the acrylic polymer by dispersing an acrylic polymer in water through homogenization or simple mixing, 2) add the water soluble components of the composition into the first premix, 3) prepare a second premix of the particulate materials, 4) combine the first and the second premix, 5) add pH adjuster to the combined mixture of the first and the second premix. The pH adjuster can also he added in any of steps 1)-4).

In one embodiment where the composition comprises humectants, the humectants can be mixed with the particulate materials during preparation of the second premix.

In another embodiment where a perfume or small amount of other oil-soluble ingredients is included in the present composition, another premix of the oil-soluble ingredients and emulsifiers can be prepared. This premix can he added to either the first or the second premix or added simultaneously during the combination of the first and the second premix.

Method of Use

The present composition can he suitably used for topical application on mammalian keratinous tissue, including, hut are not. limited to skin care composition, hair care composition and the like. Suitable skin care composition includes for example a toner.

Array of Personal Care Compositions

The present invention also relates to an array of two or more low viscosity personal care compositions. In the array, one of the compositions comprises suspended particulate materials at a level different from that in the other personal care compositions of the present invention. The varied levels of particulate materials provide varied translucency levels of the personal care composition from slightly translucent to near opaque. The array of compositions with varied translucency can he displayed on shelf together and/or sold together,

Methods of Measurement

Viscosity Measurement

The product viscosity can he measured by a commercially available viscometer, e.g., BROOKFIELD DV II+Viscometer using a UL adapter and an LV spindle set. For measurement of a viscosity which is higher than 12 mPa·s, a rotation speed of 5 rpm should he used. For measurement of viscosity which is low, e.g., 1-12 mPa·s, a rotation speed of 50 rpm should he used.

Yield Value Measurement

The yield value can be measured by using commercial available rheometers. The present invention measures the yield value by using a Modular Compact Rheometer PHYSICA MCR 300 with sensor TEZ 150P-C commercially available from Anton Paar GmbH. The flow test analysis software is Physica RheoPlus, and the shear rate applied in the measurement is from 0.01 sec⁻¹ to 10 sec⁻¹.

Stability Measurement

The accelerated stability test is conducted at 40° C. and 75% relative humidity. The success criterion for the stability test is that the test product remains stable for at least one week with essentially no particulate materials noticeably settling to the bottom or floating to the top of the product via naked eve observation. “Essentially no” means less than about 0.1%, preferably less than 0.05% of the suspended particulate materials settle to the bottom or float to the top of the product. The observation can he facilitated by putting the test product in a transparent container and directing light in a perpendicular direction toward the container.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Examples A-M

Examples A-F represent. a number of screen solutions comprising acrylic polymers selected for suspending particulate materials in the present low viscosity personal care compositions. Examples G-M represent a number of screen solutions comprising different polymers (other acrylic polymer and non-acrylic polymers) not selected for suspending particulate materials in the present low viscosity personal care compositions.

Each of the screen solutions for Examples A-M is prepared as follows. First, a polymer solution is prepared by dispersing each of the polymers in water with the pH adjusted to about 6. Then, 0.2% polymethylsilsesquioxane and 0.1% NaCl is added into the polymer solution and mixed well to arrive at a screen solution.

Each of the polymers used in Examples A-M is described in the following Table 1. Also included in Table 1 are the rheology parameters, i.e., viscosity and yield value of each screen solution, and the suspension stability results of each screen solution.

TABLE 1
Polymers in Examples A-M and particulate ulate material suspension stability results

| Examples | A | B | C | D | E | F | G |
|---|---|---|---|---|---|---|---|
| Polymers | Pemulen TR-2 | Stabylen 30 | Carbopol 981 | Pemulen TR-1 | Carbopol 980 | Carbopol 1382 | Carbopol |
| | | | | | | | Ultrez 10 |
| INCI of | Acrylates/ | Acrylates/ | Carbomer | Acrylates/ | | Acrylates/ | carbomer |
| polymer | C10-30 Alkyl | Vinyl | | C10-30 Alkyl | | C10-30 Alkyl | |
| | Acrylate | Isodecanoate | | Acrylate | | Acrylate | |
| | Crosspolymer | Crosspolymer | | Crosspolymer | | Crosspolymer | |
| PH | 6.069 | 6.183 | 5.969 | 6.045 | 6.064 | 6.102 | 6.089 |
| Viscosity | 469.1 | 515.9 | 441.5 | 485.9 | 507.5 | 541.1 | 495.5 |
| Yield | 0.121 | 0.122 | 0.142 | 0.112 | 0.135 | 0.121 | 0.101 |
| value | | | | | | | |
| (Pa) | | | | | | | |
| Stability | Y | Y | Y | Y | Y | Y | N |

| Examples | H | I | J | K | L | M |
|---|---|---|---|---|---|---|
| Polymers | Carbopol | Carbopol | Carbopol 954 | Nastrol 250 | Xanthan | Sepigel 305 |
| | Ultrez 20 | Ultrez 21 | | HHR | Gμm | |
| INCI of | Acrylates/ | Acrylates/ | carbomer | Hydroxyethyl | Xanthan | polyacrylamide |
| polymer | C10-30 Alkyl | C10-30 Alkyl | | cellulose | Gμm | |
| | Acrylate | Acrylate | | | | |
| | Crosspolymer | Crosspolymer | | | | |
| PH | 6.172 | 6.145 | 6.081 | 5.95 | 5.923 | 6.004 |
| Viscosity | 494.3 | 526.7 | 495.5 | 476.3 | 488.7 | 517.1 |
| Yield | 0.080 | 0.093 | 0.061 | N/A | 0.247 | 0.0566 |
| value | | | | | | |
| (Pa) | | | | | | |
| Stability | N | N | N | N | N | N |
| (Y/N) | | | | | | |

The selection solutions of Examples A-F which comprise acrylic polymers (certain species of carbomer and acrylates/C₁₀₋₃₀alkyl acrylate crosspolymer) provide a selection yield value of at least 0.11Pa and exhibit the requisite suspension stability.

The selection solutions of Examples G-J which comprise different acrylic polymers (the other species of carbomer and acrylates/C₁₀₋₃₀ alkyl acrylate crosspolymer) provide a selection less than 0.11 Pa and do not exhibit the requisite suspension stability.

The Examples K-M. solutions which comprise non-acrylic polymers (e.g., cellulose, gums, polyacrylamide) provide a selection yield value of either higher or lower than 0.11 Pa do not exhibit the requisite suspension stability. Both the internal structure and the yield value threshold provided by the polymers arc believed to he important in suspending the particulate materials. Without being bound by theory, it is believed that the non-acrylic polymers, though they provide a selection yield value of higher than 0.11 Pa. they do not build a cross-linked internal structure of the selection like the select acrylic polymer do.

### Examples 1-8 and Comparative Examples 1-3

Examples 1-8 are the low viscosity personal care composition according to the present invention, while Comparative Examples 1-3 are compositions which fall outside of the present invention. Examples 1-8 and Comparative Examples 1-3 compositions can he suitably made as described below:

1) A first premix of the polymer is prepared by dispersing each of the acrylic polymers (component # 16-20) of Examples 1-8 and Comparative Examples 1-3 in de-ionized water (component # 1).

2) The water soluble components (component # 2-7) are added and mixed well with the polymer.

3) The pH adjuster (component is then added into the above mixture to neutralize the polymer,

4) A second premix of the particulate materials is prepared by blending glycols (component # 8, #10) and the particulate materials (component # 11 -15) in Examples 1-8 and Comparative Examples 1-3.

5) The second premix is then added into the first premix and then they are mixed well using conventional mixing techniques.

6) The emulsifier (component # 9 is added into the mixture achieved in step 3 and they are mixed well until the particulate materials are well dispersed.

The prepared compositions are then tested for stability according to the accelerated stability test described herein.

TABLE 2
Examples 1-10

| Compo- | Component | | | | | | | | | Com. | Com. | Com. |
|---|---|---|---|---|---|---|---|---|---|---|---|---|
| nen t# | Name | Ex. 1 | Ex. 2 | Ex. 3 | Ex. 4 | Ex. 5 | Ex. 6 | Ex. 7 | Ex. 8 | Ex. 1 | Ex. 2 | Ex. 3 |
| 1 | De-ionized Water | Qsp | Qsp | Qsp | Qsp | Qsp | Qsp | Qsp | Qsp | Qsp | Qsp | Qsp |
| 2 | Sodium Chloride | 0.1 | 0.1 | 0.1 | 0.1 | 0.1 | 0.1 | 0.1 | 0.1 | 0.1 | 0.1 | 0.1 |
| *3 | Niacinamide | 5 | 5 | 5 | 5 | 5 | 5 | 5 | 5 | 5 | 5 | 5 |
| 4 | Sodium Benzoate | 0.1 | 0.1 | 0.1 | 0.1 | 0.1 | 0.1 | 0.1 | 0.1 | 0.1 | 0.1 | 0.1 |
| 5 | Disodium EDTA | 0.1 | 0.1 | 0.1 | 0.1 | 0.1 | 0.1 | 0.1 | 0.1 | 0.1 | 0.1 | 0.1 |
| 6 | Benzyl Alcohol | 0.2 | 0.2 | 0.2 | 0.2 | 0.2 | 0.2 | 0.2 | 0.2 | 0.2 | 0.2 | 0.2 |
| 7 | Phenoxyethanol | 0.25 | 0.25 | 0.25 | 0.25 | 0.25 | 0.25 | 0.25 | 0.25 | 0.25 | 0.25 | 0.25 |
| 8 | Glycerol | 5 | 5 | 5 | 5 | 5 | 5 | 5 | 5 | 5 | 5 | 5 |
| 9 | Gkycereth-25 | 0.25 | 0.25 | 0.25 | 0.25 | 0.25 | 0.25 | 0.25 | 0.25 | 0.25 | 0.25 | 0.25 |
| | PCA Isostearate | | | | | | | | | | | |
| 10 | Butylene Glycol | 6 | 6 | 6 | 6 | 6 | 6 | 6 | 6 | 6 | 6 | 6 |
| 11* | C7F-600 | 1 | — | — | — | — | 1 | 1 | 1 | 1 | 1 | 1 |
| 12* | Tospearl 145A | — | 1 | — | — | — | — | — | — | — | — | — |
| 13* | Tospearl 150KA | — | — | 1 | — | — | — | — | — | — | — | — |
| *14* | Tospearl AQ | — | — | — | 1 | — | — | — | — | — | — | — |
| 15* | Nylon 12 | — | — | — | — | 1 | — | — | — | — | — | — |
| 16* | Stabylen-30 | 0.2 | 0.2 | 0.2 | 0.2 | 0.2 | | 0.1 | — | — | — | — |
| 17* | Pemulen TR-2 | — | — | — | — | — | 0.2 | 0.1 | — | — | — | — |
| 18* | Carbopol 981 | — | — | — | — | — | — | — | 0.2 | — | — | — |
| 19* | Carbopol Ultrez | — | — | — | — | — | — | — | — | 0.2 | 0.29 | — |
| | 10 | | | | | | | | | | | |
| *20* | Carbopol Ultrez | — | — | — | — | — | — | — | — | — | — | 0.2 |
| | 20 | | | | | | | | | | | |
| 21 | Triethanolamine | 0.2 | 0.2 | 0.2 | 0.2 | 0.2 | 0.2 | 0.2 | 0.2 | 0.2 | 0.2 | 0.2 |
| | Viscosity (mPa.s) | 515.9 | 471.5 | 483.5 | 514.7 | 477.5 | 927.4 | 641.9 | 601.1 | 18.0 | 495.5 | 256.7 |
| | pH | 6.183 | 6.168 | 6.165 | 6.227 | 6.174 | 6.124 | 6.166 | 6.030 | 6.078 | 6.089 | 6.102 |
| | Stability (Y/N) | Y | Y | Y | Y | Y | Y | Y | Y | N | N | N |

*11 CF600, Polymethylsilsesquioxane, available from Momentive
*12 Tospearl 145A, Polymethylsilsesquioxane, available from Momentive
*13 Tospearl 150KA, Polymethylsilsesquioxane, available from Momentive
*14 Tospearl AQ, a mixture of polymethylsilsesquioxane, polyquaternium-7. PEG-7 glyceryl cocoate and methylsilanol tri-PEG-8 glyceryl cocoate, from Momentive
*15 Nylon 12, from IMCD UK Ltd.
*16 Stabylen-30, Acrylates/Vinyl Isodecanoate Crosspolymer, from 3V Inc.
*17 PemulEn TR-2, Acrylates/C10-30 Alkyl Acrylate Crosspolymer, front Lubrizol
*18 Carbopol 981, Carbomer, from Lubrizol
*19 Carbopol Ultrez 10, Carbomer, from Lubrizol
*20 Carbopol Ultrez 10, Acrylates/C10-30 Alkyl Acrylate Crosspolymer, from Lubrizol

The compositions of Examples 1-8 compositions have a viscosity ranging from about 470 mPa·s to about 930 mPa·s. Through using the selected acrylic polymers or mixture of acrylic polymers which have a selection yield value of at least 0.11 Pa, these compositions exhibit stability as defined herein.

The compositions of Comparative Examples 1-3 have a viscosity ranging from 18 to about 500 mPa·s. These compositions comprise acrylic polymers which have a selection yield value of less than 0.11 Pa and do not exhibit stability as defined herein.

The dimensions and values disclosed herein are not to he understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would he obvious to those skilled in the art that various other changes and modifications can be made without departing from the spirit and scope of the invention. It is therefore intended to cover in the appended claims all such changes and modifications that are within the scope of this invention.

What is claimed is:

1. A low viscosity personal care composition comprising,
a) from about 10% to about 99% of water,
b) from about 0.01% to about 2% of a suspended particulate material,
c) from about 0.01% to about 5% of an acrylic polymer,
wherein said composition has a viscosity of from about 80 to about 1000 mPa·s, and a pH of from about 4 to about 9, wherein said acrylic polymer provide a selection yield value of at least 0.11 Pa.

2. The personal care composition of claim 1, wherein said suspended particulate material comprises particles having a median particle size of about 2 μm to about 15 μm.

3. The personal care composition of claim 1 or 2, wherein said suspended particulate material comprises particles having a specific density of from about 0.8 cm³ to about 1.3 cm³.

4. The personal care composition of claim 1, wherein said acrylic polymer provides a selection yield value of at least 0.12 Pa.

5. The personal care composition of claim 1, wherein said acrylic polymer is an acrylates/vinyl isodecanoate cross polymer.

6. The personal care composition of claim 1, which further comprises from about 0.01% to about 10% of a viscosity reducing agent.

7. The personal care composition of claim 6, wherein said viscosity reducing agent is a dissociable active agent.

8. The personal care composition of claim 7, wherein said dissociable active agent is selected from a group consisting of dipotassium glycyrrhizinate, undecylenoyl phenylalanine, sodium ascorbyl phosphate, hexamidine diisethionate, and combinations thereof.

9. The personal care composition of claim 1, wherein said viscosity is from about 200 to about 600 mPa·s.

10. The personal care composition of claim 1. wherein said pH is from about 5 to about 7.

11. The personal care composition of claim 1 which comprises no more than about 10% of an emulsifier.

12. The personal care composition of claim 1 which further comprises up to about 30% of a humectant.

13. The personal care composition of claim 12, wherein said humectant is a polyhydroxy alcohol.

14. The personal care composition of claim 1, wherein said suspended particulate material is selected from a group consisting of polymethylsilsesquioxane, nylon, polyethylene, and combinations thereof.

15. A low viscosity personal care composition comprising:
e) from about 50 % to about 99% of water,
f) from about 0.01% to about 20% of polymethylsilsesquioxane,
g) from about 0.01% to about 5% of an acrylates/vinyl isodecanoate cross polymer,
h) from about 0.01% to about 10% of a viscosity reducing agent, which is a dissociable active agent, wherein said composition has a viscosity of from about 200 to about 600 mPa·s, and a pH of from about 5 to about 7.

16. The personal care composition of claim 15, wherein said dissociable active agent is selected from a group consisting of dipotassium glycyrrhizinate, undecylenoyl phenylalanine, sodium ascorbyl phosphate, hexamidine diisethionate, and combinations thereof.

17. An array of two or more low viscosity personal care compositions according to any of claims 1-16, wherein one of said personal care compositions comprises said particulate material at a level different from those in the other personal care compositions.
